(19) United States
(12) Patent Application Publication
(10) Pub. No.: US 2009/0113759 A1
(43) Pub. Date: May 7, 2009

(54) THERAPEUTIC FOOTWEAR

(76) Inventor: Chele Suzanne Heid, Greenwood, IN (US)

Correspondence Address: Chele S. Heid, 754 Valley Way Rd., Greenwood, IN, 46142, US

(21) Appl. No.: 11/935,380

(22) Filed: Nov. 5, 2007

(51) Int. Cl. A43B 7/16; A43B 7/14; A43B 7/22; A61F 5/14

(52) U.S. Cl. 36/92; 36/140; 36/88; 36/91

(57) ABSTRACT

The present invention generally relates to therapeutic footwear such as sandals, conventional shoes, and corrective inserts that may be inserted into conventional shoes. Specifically, the therapeutic footwear may be used for maintaining, correcting, or repositioning the great toe to decrease pressure on the first metatarsal-phalangeal joint or bunion. Additional therapeutic features may include spreading of the wearer's toes, supporting the wearer's arch to control excessive pronation, and providing sustained heel cord stretch at rest, during ambulation, pedicure, and before, during, or after medical treatment.

### BACKGROUND AND SUMMARY OF THE INVENTION

Foot pain, bunions, stiff Achilles tendons/heel cords, and plantar fasciitis have long been problems suffered by many, especially women and most especially women who wear high heels. Inherent in high heels is the problem of tight heel cords due to the heel being elevated above the forefoot thus placing the heel cords in a shortened position. Also problematic in this type of footwear is the forward force placed on the foot, from heel to toes, thereby increasing the pressure on the forefoot and the metatarsal heads while forcing the metatarsal-phalangeal joints into an unnaturally increased dorsiflexed and adducted position. Additionally, many high heeled shoes are designed with an ever narrowing toe box which often tapers to a point thus diminishing any normal physiological space between the toes and causing the great toe to deviate laterally. With frequent use of this type of footwear come the aforementioned problems. These problems can also be caused by a number of other factors not limited to a pronated foot, genetics, lifestyle, and activity type and level.

There are many products available to effect a separation between the toes (inserts, braces, and other accessories,) however none of these products incorporates these features contiguously into a footwear for daily wear. Use of aforementioned accessories can be labor intensive in applying the device to the toes, foot, or shoe. They may be uncomfortable or impossible to fit into a specific type of shoe as well as unattractive as evidenced by the bunion brace being visible when worn with several shoe styles. Treatments also exist to address pain associated with the above conditions including physical therapy, medication via injection or by mouth, and cryotherapy. Surgery has also been employed to correct the misalignment of the great toe. The present disclosure addresses the root cause of bunion pain, relieving pain without the complications and side effects of surgical intervention.

One embodiment of the present disclosure includes a therapeutic footwear for supporting a wearer's foot having a heel portion, an arch portion, and a plurality of toes including a great toe, the footwear including a horizontally extending base having first and second spaced apart ends, the first end adapted to support the heel portion of the wearer's foot, the second end adapted to support the plurality of toes, and an adjustable strap including an adjustment mechanism and first and second portions, the first portion coupled to the second end of the base and adapted to secure the great toe, the second portion adapted to secure the adjustable strap to the base, the adjustment mechanism adapted to pull the great toe away from the other toes of the plurality of toes.

Another embodiment of the present disclosure includes a therapeutic footwear for supporting a wearer's foot having a heel portion, an arch portion, and a plurality of toes including a great toe, the footwear including a horizontally extending base having first and second spaced apart ends, a passage, a recessed area, and a slot, the first end adapted to support the heel portion of the wearer's foot, the second end adapted to support the plurality of toes, the passage extending laterally through the base, the recessed area positioned on the second end of the base, the slot including a first opening on an upper side of the base and second opening on lateral side of the base, an adjustable strap including an adjustment mechanism and first and second portions, the first portion coupled to the recessed area of the second end of the base and adapted to secure the great toe, the second portion extending through the passage and the slot to secure the adjustable strap to the base, the adjustment mechanism adapted to pull the great toe away from the other toes of the plurality of toes, and a second strap coupled to the second portion of the adjustable strap and the second portion of the base, the second strap including a plurality of leaders on a first end and an adjustable portion on a second end, the second end extending around the adjustable strap to secure the second strap around the heel portion, the plurality of leaders coupled to the second portion of the base.

Yet another embodiment of the present disclosure includes a footwear insert for providing therapeutic support to a wearer's toes inside the wearer's conventional shoe, the footwear insert including a horizontally extending base having a heel end and a toe end, the base extending horizontally within the conventional shoe, and a band coupled to the toe end of the base, the band adapted to apply a laterally inward pressure to a great toe of the wearer's toes.

### BRIEF DESCRIPTION OF THE DRAWINGS

The detailed description of the drawings particularly refers to the accompanying figures in which:

FIG. 1 is a perspective view one embodiment of a therapeutic footwear;

FIG. 2 is a top view of the therapeutic footwear shown in FIG. 1;

FIG. 3 is a cross-sectional view taken along the front of the therapeutic footwear shown in FIGS. 1 and 2;

FIG. 4 is a cross-sectional view taken along the length of the therapeutic footwear shown in FIGS. 1 through 3;

FIG. 5 is a medial side view of the therapeutic footwear shown in FIGS. 1 through 4;

FIG. 6 is a perspective view of another embodiment of a therapeutic footwear; and

FIG. 7 is a perspective view of a conventional shoe having a therapeutic features.

### DETAILED DESCRIPTION OF THE DRAWINGS

The embodiments of the invention described herein are not intended to be exhaustive or to limit the invention to the precise forms disclosed. Rather, the embodiments selected for description have been chosen to enable one skilled in the art to practice the invention.

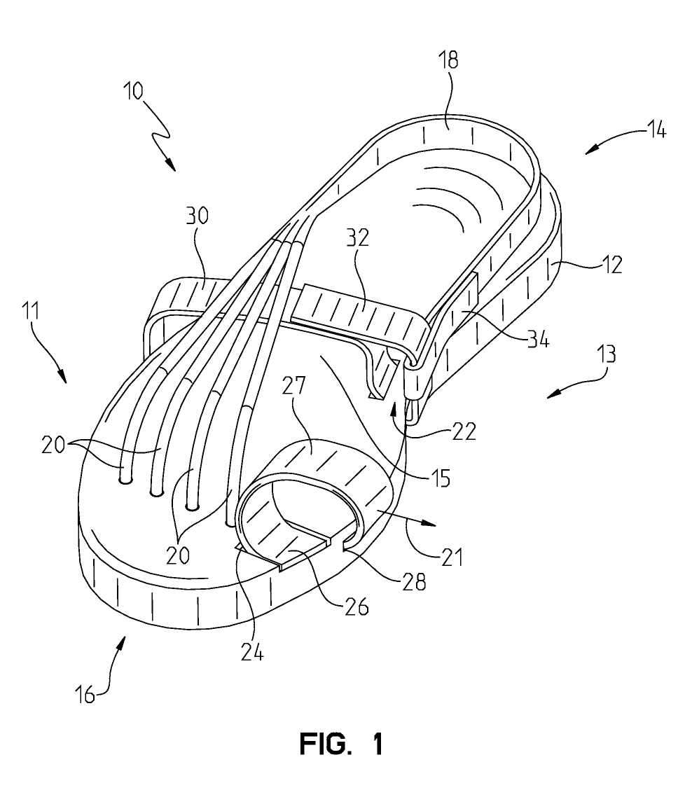

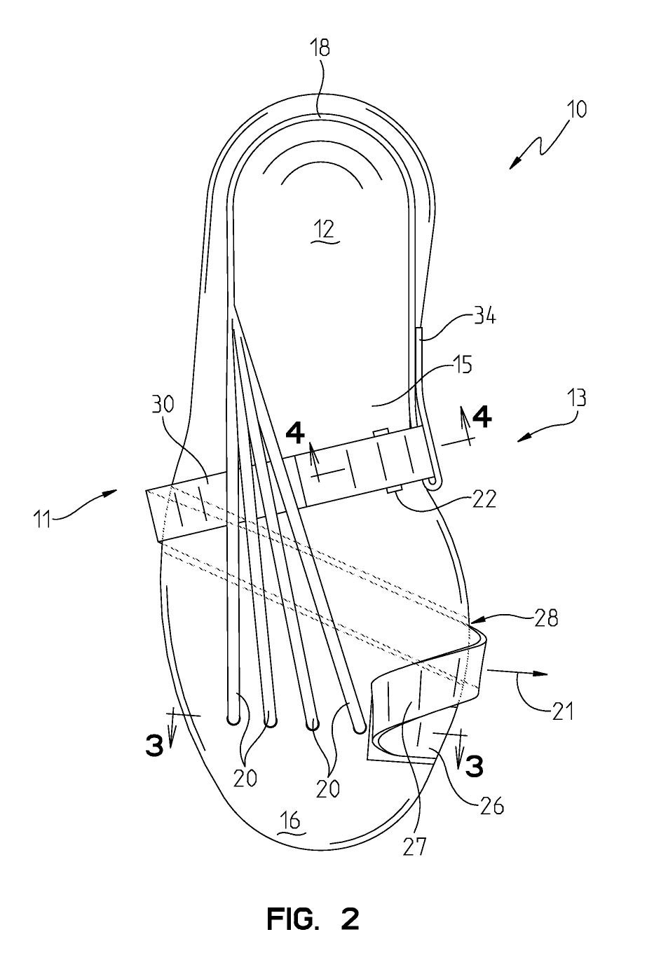

Referring to FIGS. 1 and 2, a therapeutic footwear 10 is represented. Footwear 10 is designed to prevent hallux valgus or bunions by maintaining proper great toe alignment and stabilizing the great toe. Footwear 10 also treats existing hallux valgus or bunions and provides pre-surgical and post-bunionectomy/great toe realignment surgery by supporting the great toe, assisting in spreading a wearer's toes, providing support to the wearer's arch to limit excessive pronation, and providing a sustained gentle stretch to the wearer's heel cords, at rest, during ambulation, during pedicure, and before, during, or after medical treatment.

Footwear 10 provides therapeutic relief to the feet of the wearer. Specifically, footwear 10 provides relief for wearers having a bunion on the great toe (commonly referred to as “the big toe” or “first toe”). Footwear 10 may be used to apply a medial force to the great toe of the wearer's foot to force the great toe into the anatomically correct position to relieve bunion pain. For simplicity, footwear 10 is illustrated and explained as only the “right” side shoe or footwear. It should be understood that the “left” side shoe or footwear is a mirror image of footwear 10 shown in FIG. 1.

Footwear 10 includes a horizontally extending base 14 having first end 12 and second end 16. Base 14 includes medial side 13 and lateral side 11. First end 12 supports the heel portion of the wearer's foot and second end 16 supports the wearer's toes. The arch of the wearer's foot is supported by section 15 of base 12. Base 12 also includes passage 28 and slot 22. Passage 28 extends diagonally through the interior of base 12 from medial side 12 to lateral side 11. Passage 28 is formed to accept adjustable strap 30. Slot 22, shown in FIG. 4, has a first opening on the upper surface of base 12 and a second opening on medial side 13 of base 12. Slot 22 is also formed to accept strap 30.

Adjustable strap 30 includes first end 26 and second end 32. First end 26 of strap 20 is coupled to recessed area 24 of base 12. When the wearer's foot enters footwear 10, the great toe slides into loop 27 which is formed by strap 30. Strap 30 passes from medial side 13 of base 12 through passage 28 and exits passage 28 on lateral side 11 of base 12. Strap 30 then extends upward and over the top of the wearer's foot (not shown) and extends downward through the first opening of slot 22 in the upper surface of base 12. Strap 30 exits slot 22 through the second opening on medial side 13 of base 12.

The wearer may adjust medial force 21 on the great toe by adjusting an adjustment mechanism on adjustable strap 30. In the illustrative embodiment, second end 32 of adjustable strap 30 includes Velcro and couples to a corresponding Velcro surface on the upper surface of strap 30 over the top of the wearers foot. Adjustable strap 30 may include any suitable adjustment mechanism such as Velcro, snaps, pins, or buckles. To apply more medial force on the great toe, second end 32 of adjustable strap 30 is tightened by pulling it toward lateral side 11 of base 12 and secured by the adjustment mechanism of adjustable strap 30. Conversely, to lessen medial pressure 21 on the great toe, second end 32 of adjustable strap 30 is loosened to allow it to move toward medial side 13 of base 12 until it is secured by the adjustment mechanism of adjustable strap 30.

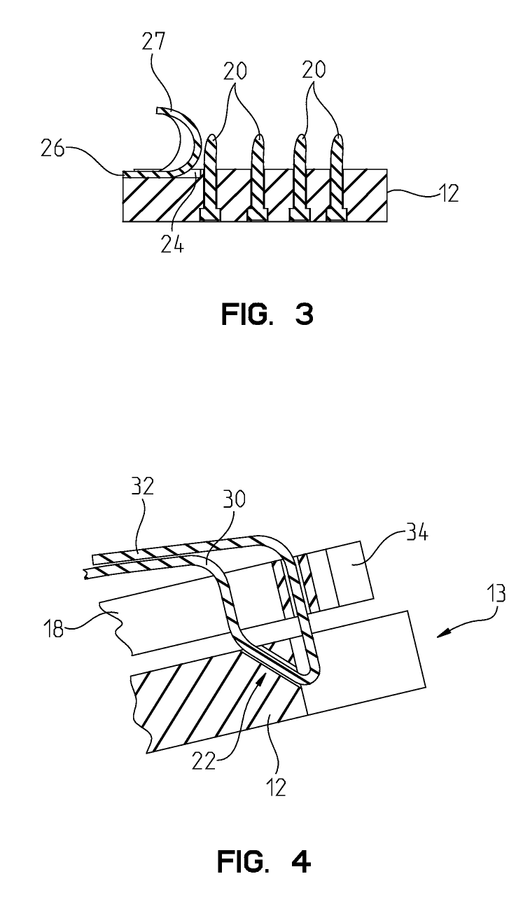

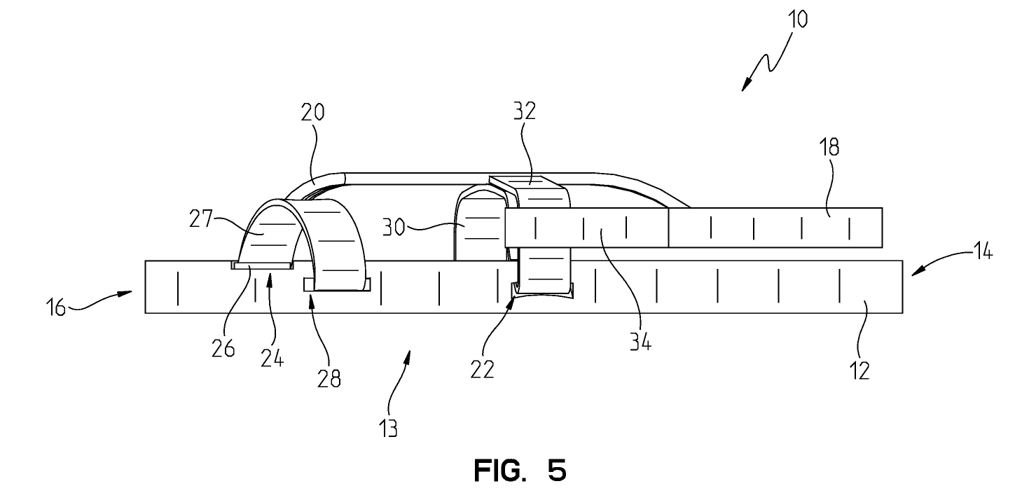

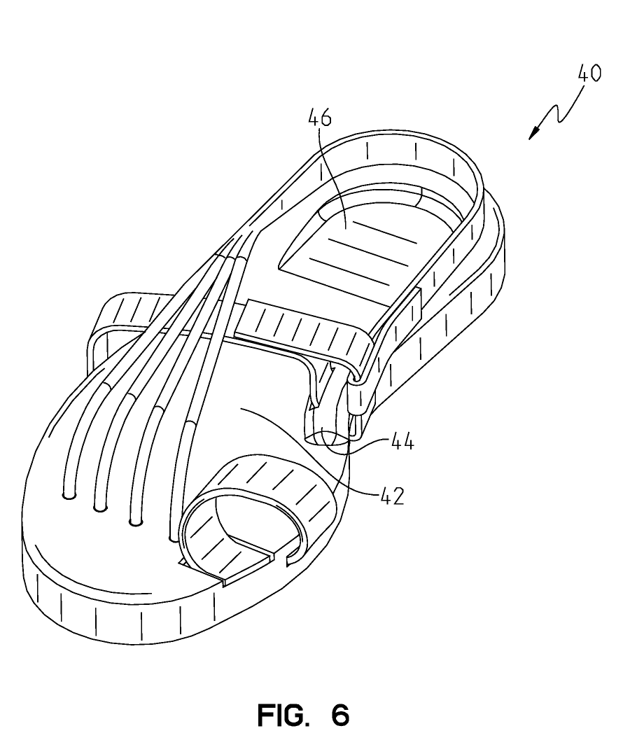

Footwear 10 also includes second strap 18 to secure footwear 10 to the wearer's heel portion. Second strap 18 includes first end 34 which is looped around adjustable strap 30 on medial side 13 of base 12 in the illustrative embodiment. Second strap 18 also includes plurality of leaders 20 which couple to second end 16 of base 12, shown in FIG. 3. Leaders 20 are coupled to base 12 at positions corresponding to the spacing between the wearer's toes to separate the wearer's toes when the foot is inserted into footwear 10. In other embodiments, leaders 20 may be replaced by a strap connected only to loop 27 of adjustable strap 30.

Second strap 18 may include an adjustment mechanism similar to adjustable strap 30. In the illustrative embodiment, first end 32 includes a Velcro surface corresponding to a second Velcro surface on second strap 18. In the illustrative embodiment shown in FIGS. 1 and 2, the wearer may secure footwear 10 to the foot by pulling first end 34 through the loop formed by adjustable strap 30 and securing the opposing Velcro surfaces on second strap 18. As discussed above, the adjustment mechanism of second strap 18 may be any suitable fastening system including Velcro, snaps, pins, or buckles.

The components of footwear 10 may be formed from leather, rubber, synthetic material, cloth, sheepskin, canvas, polyester, polyurethane, nylon, polypropylene, neoprene, mesh, recycled materials, plastic, or any other suitable material. Additionally, straps 18 and 30 may be formed from any suitable elastic or inelastic materials such as nylon, cloth, leather, plastic, rubber, polyester, or canvas.

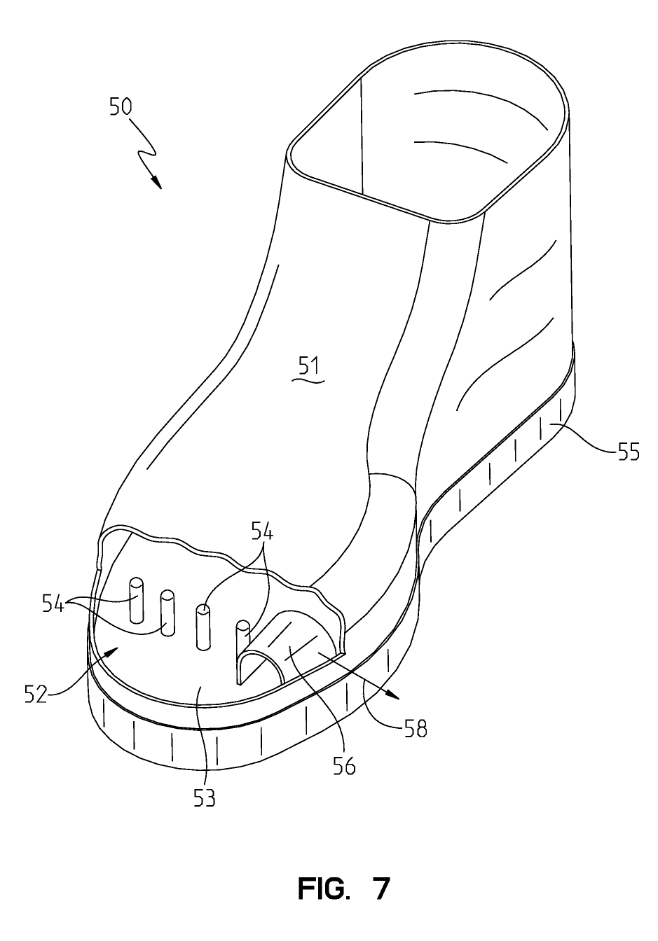

Another embodiment of a therapeutic footwear is shown in FIG. 6. Therapeutic footwear 40 is similar to therapeutic footwear 10 shown above with several exceptions. Horizontally extending base 42 of footwear 40 includes recessed area 46 below the wearer's heel portion. Recessed area 46 creates a “negative heel” which may provide a gentle calf stretch. Additionally recessed area 46 may provide relief to the toes of the wearer by reducing the downward and forward pressure of the wearer's body on the tarsal joints, especially after a period of wearing shoes have a positive heel, i.e. high heeled shoes. Therapeutic footwear 40 may also include arch support 44 formed within or coupled to base 42. Arch support 44 may be formed to any suitable height or width to provide arch support and pain relief to the wearer's arch.

One embodiment of a conventional shoe having therapeutic features is shown in FIG. 7. A conventional shoe or footwear 50 is shown including sole 55 and covering surface 51. Examples of conventional shoes may include boots, tennis shoes, loafers, slippers, moccasins, clogs, or any other suitable closed toe or semi-closed toe shoe. Therapeutic portion 52 is shown within conventional shoe 50. Therapeutic portion 52 includes base 53, extensions 54, and band 56. Base 53 extends the entire length of the interior cavity of shoe 50 and may be formed of a rigid or flexible material. Extensions 54 are positioned to separate the wearer's toes when the foot is inserted into shoe 50. When the foot is inserted into shoe 50, the great toe enters band 50 which applies medial force 58 to pull the great toe toward the center line of the wearer's body to relieve bunion pain. Band 56 may be formed from an elastic or inelastic material. In other embodiment, extensions 54 may be absent.

In another embodiment, similar to footwear 50, therapeutic portion 52 may be formed as an insert incorporating extensions 54 and band 56 of therapeutic portion 52, as shown in FIG. 7. In this embodiment, band 56 may include an adjustment means such as a Velcro portion to allow the wearer to vary medial force 58 on the great toe. The adjustment means may include a corresponding Velcro portion positioned on the bottom side of the insert to couple to the Velcro portion affixed to band 56. The adjustment means may include any other suitable fastener such as buckles or buttons.

Although the invention has been described in detail with reference to certain preferred embodiments, variations and modifications exist within the spirit and scope of the invention as described and defined in the following claims.

1. A therapeutic footwear for supporting a wearers foot having a heel portion, an arch portion, and a plurality of toes including a great toe, the footwear including:
a horizontally extending base having first and second spaced apart ends, the first end adapted to support the heel portion of the wearer's foot, the second end adapted to support the plurality of toes; and
an adjustable strap including an adjustment mechanism and first and second portions, the first portion coupled to the second end of the base and adapted to secure the great toe, the second portion adapted to secure the adjustable strap to the base, the adjustment mechanism adapted to pull the great toe away from the other toes of the plurality of toes.

2. The therapeutic footwear of claim 1, wherein the base includes a passage extending through the base and the adjustable strap extends through the passage.

3. The therapeutic footwear of claim 2, wherein the base includes a slot having first opening on an upper surface of the base and a second opening on a lateral side of the base.

4. The therapeutic footwear of claim 3, wherein the second end of the adjustable strap extends through the first and second openings of the slot to secure the adjustable strap to the base.

5. The therapeutic footwear of claim 4, wherein the adjustment mechanism is one Velcro, a snap, a buckle, a latch, a button, and a zipper.

6. The therapeutic footwear of claim 1, further comprising a second strap adapted to secure the footwear to the heel portion of the wearer's foot.

7. The therapeutic footwear of claim 6, wherein the second strap includes a plurality of leaders coupled to the second end of the base, the plurality of leaders coupled to the second end of the base at a plurality of positions corresponding to a spacing between the plurality of toes.

8. The therapeutic footwear of claim 6, wherein the second strap includes an adjustable portion.

9. The therapeutic footwear of claim 8, wherein the second strap is coupled to the adjustable strap.

10. The therapeutic footwear of claim 1, wherein the first end of the base includes a recessed area adapted to support the heel.

11. The therapeutic footwear of claim 1, further comprising an arch support adapted to provide support to the arch.

12. A therapeutic footwear for supporting a wearer's foot having a heel portion, an arch portion, and a plurality of toes including a great toe, the footwear including:
a horizontally extending base having first and second spaced apart ends, a passage, a recessed area, and a slot, the first end adapted to support the heel portion of the wearer's foot, the second end adapted to support the plurality of toes, the passage extending laterally through the base, the recessed area positioned on the second end of the base, the slot including a first opening on an upper side of the base and second opening on lateral side of the base;
an adjustable strap including an adjustment mechanism and first and second portions, the first portion coupled to the recessed area of the second end of the base and adapted to secure the great toe, the second portion extending through the passage and the slot to secure the adjustable strap to the base, the adjustment mechanism adapted to pull the great toe away from the other toes of the plurality of toes; and
a second strap coupled to the second portion of the adjustable strap and the second portion of the base, the second strap including a plurality of leaders on a first end and an adjustable portion on a second end, the second end extending around the adjustable strap to secure the second strap around the heel portion, the plurality of leaders coupled to the second portion of the base.

13. The therapeutic footwear of claim 12, wherein the each of the plurality of leaders is positioned between each one of the plurality of toes.

14. The therapeutic footwear of claim 12, wherein the adjustment mechanism of the adjustable strap is one of Velcro, snaps, pins, a buckle, a latch, a button, and a zipper.

15. The therapeutic footwear of claim 12, wherein adjustable portion of the second strap is one of Velcro, snaps, pins, a buckle, a latch, a button, and a zipper.

16. The therapeutic footwear of claim 12, wherein first portion of the base includes a recessed area adapted to support the heel of the wearer.

17. The therapeutic footwear of claim 12, further comprising an arch support adapted to provide support to the arch.

18. A footwear insert for providing therapeutic support to a wearer's toes inside the wearer's conventional shoe, the footwear insert including:
a horizontally extending base having a heel end and a toe end, the base extending horizontally within the conventional shoe; and
a baud coupled to the toe end of the base, the band adapted to apply a laterally inward pressure to a great toe of the wearer's toes.

19. The footwear insert of claim 18, further comprising a plurality of extensions coupled to the base at positions between the wearer's toes.

20. The footwear insert of claim 18, wherein the band is formed of one of an elastic material and an inelastic material.
